(19) United States
(12) Patent Application Publication
(10) Pub. No.: US 2025/0329266 A1
(43) Pub. Date: Oct. 23, 2025

(54) ENVIRONMENTAL SIGNAL RECOGNITION TRAINING

(71) Applicant: Cochlear Limited, Macquarie University, NSW (AU)

(72) Inventors: Janette OLIVER, Mount Pleasant, NSW (AU); Jeremy Leslie SWANSON, Sunnyvale, CA (US)

(21) Appl. No.: 18/869,091

(22) PCT Filed: May 25, 2023
(86) PCT No.: PCT/IB2023/055382

§ 371 Date: Nov. 25, 2024

Related U.S. Application Data
(60) Provisional application No. 63/347,654, filed on Jun. 1, 2022.

(51) Int. Cl. G09B 5/06; A61N 1/05

(52) U.S. Cl. CPC G09B 5/06; A61N 1/0541

(57) ABSTRACT

Presented herein are techniques for training a medical device (e.g., hearing device) user to correctly perceive environmental signals, such as environmental sounds. In certain examples, the techniques presented herein provide the user with environmental signal discrimination training and/or environmental signal identification training.

### BACKGROUND

### Field of the Invention

The present invention relates generally to techniques for training medical device users to recognize environmental signals, such as environmental sounds.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: recording, at a computing device, one or more environmental signals associated with one or more ambient environments; and using the one or more environmental signals recorded at the computing device to provide environmental signal training to a medical device user.

In another aspect, a method is provided. The method comprises: providing environmental sound discrimination training to a hearing device user using a first one or more environmental sounds, wherein the first one or more environmental sounds comprise non-speech and non-musical ambient sounds; and providing environmental sound identification training to the hearing device user using a second one or more environmental sounds, wherein the second one or more environmental sounds comprise non-speech and non-musical ambient sounds.

In another aspect, one or more non-transitory computer readable storage media comprising instructions are provided. The instructions, when executed by a processor, cause the processor to: deliver one or more user interfaces enabling a user to record one or more environmental sounds; store the one or more environmental sounds in an environmental sound library; and provide environmental sound training to a hearing device user using at least one of the one or more environmental sounds stored in the environmental sound library.

In another aspect, an apparatus is provided. The apparatus comprises: one or more microphones configured to record at least one environmental sound associated with at least one ambient sound environments experienced by a hearing device user; one or more speakers; and at least one processor configured to: store the at least one environmental sound in an environmental sound library, and use the at least one environmental sound to provide environmental signal training to the hearing device user.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 1E is a schematic diagram illustrating a computing system with which aspects of the techniques presented herein can be implemented;

FIGS. 2A, 2B, and 2C are visual representations of example user interfaces that can be provided to a user to record and listen to environmental sounds, in accordance with certain embodiments presented herein;

FIGS. 3A, 3B, and 3C are visual representations of example user interfaces that can be provided to a user for initial environmental sound discrimination training, in accordance with certain embodiments presented herein;

FIGS. 4A, 4B, and 4C are visual representations of example user interfaces that can be provided to a user for initial environmental sound identification training, in accordance with certain embodiments presented herein;

FIGS. 5A and 5B are visual representations of example user interfaces that can be provided to a user for advanced environmental sound familiarization training, in accordance with certain embodiments presented herein;

FIGS. 6A, 6B, and 6C are visual representations of example user interfaces that can be provided to a user for advanced environmental sound discrimination training, in accordance with certain embodiments presented herein;

FIGS. 7A, 7B, 7C, 7D, and 7E are visual representations of example user interfaces that can be provided to a user for advanced environmental sound identification training, in accordance with certain embodiments presented herein;

FIGS. 8A, 8B, 8C, 8D, 8E, 8F, and 8G are visual representations of example user interfaces that can be provided to a user for advanced environmental sound sequencing training, in accordance with certain embodiments presented herein;

FIG. 9 is a first flowchart illustrating an example method, in accordance with certain embodiments presented herein;

FIG. 10 is a second flowchart illustrating an example method, in accordance with certain embodiments presented herein;

FIG. 11 is schematic diagram illustrating an implantable stimulation system with which aspects of the techniques presented herein can be implemented;

FIG. 12 is a schematic diagram illustrating a retinal prosthesis system with which aspects of the techniques presented herein can be implemented.

FIG. 13 is a functional block diagram of a computing environment with which aspects of the techniques presented herein can be implemented.

### DETAILED DESCRIPTION

Presented herein are techniques for training a medical device (e.g., hearing device) user to correctly perceive environmental signals, such as environmental sounds. In certain examples, the techniques presented herein provide the user with environmental signal discrimination training and/or environmental signal identification training.

Merely for ease of description, the techniques presented herein are primarily described with reference to a specific medical device system, namely a cochlear implant system. However, it is to be appreciated that the techniques presented herein can also be partially or fully implemented by other types of medical device systems. For example, the techniques presented herein can be implemented by hearing aid systems and/or auditory prosthesis systems that include one or more other types of auditory prostheses, such as cochlear implants, middle ear auditory prostheses, bone conduction devices, direct acoustic stimulators, electro-acoustic prostheses, auditory brain stimulators, combinations or variations thereof, etc. The techniques presented herein can also be implemented dedicated tinnitus therapy devices and tinnitus therapy device systems. In further embodiments, the presented herein can also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

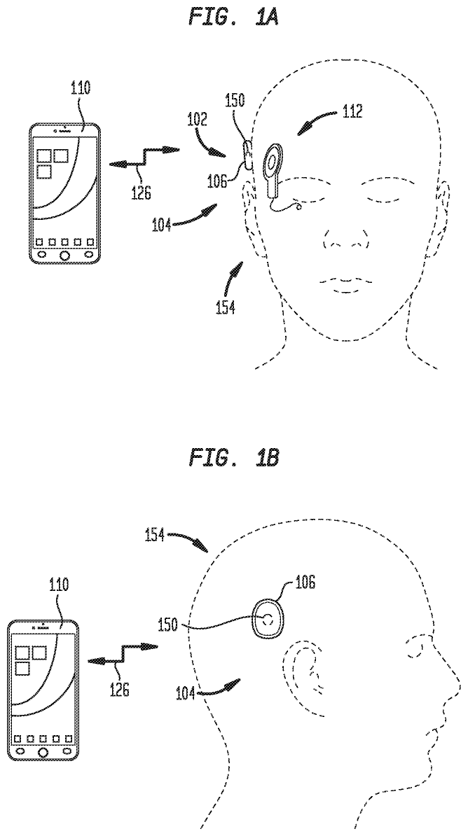

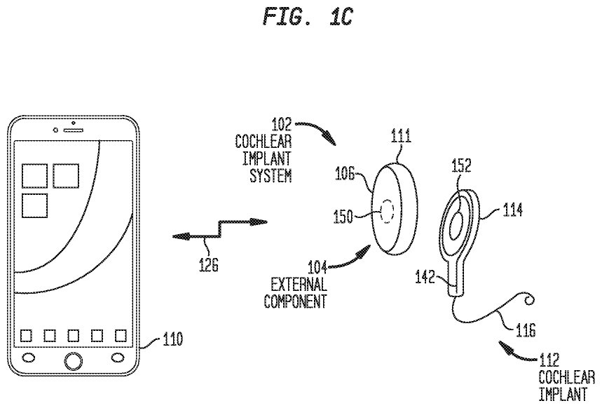

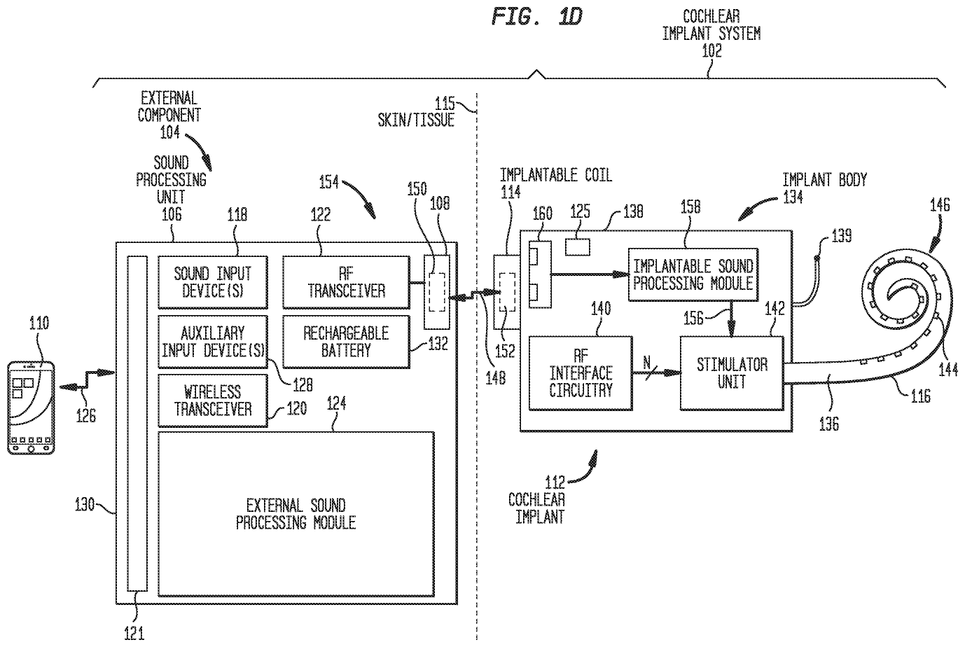

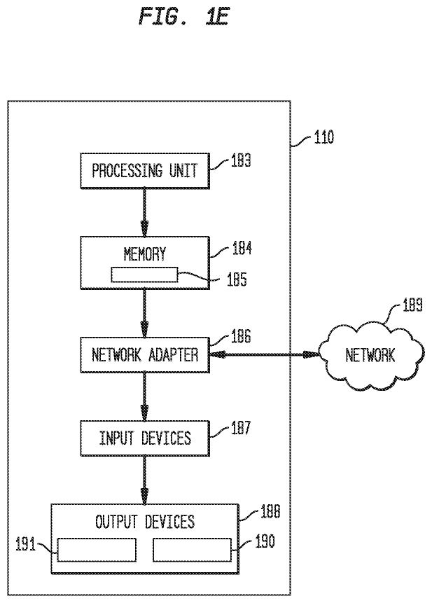

FIGS. 1A-1E illustrates an example cochlear implant system 102 with which aspects of the techniques presented herein can be implemented. The cochlear implant system 102 comprises an external component 104 and an implantable component 112. In the examples of FIGS. 1A-1E, the implantable component is sometimes referred to as a “cochlear implant.” FIG. 1A illustrates the cochlear implant 112 implanted in the head 154 of a user, while FIG. 1B is a schematic drawing of the external component 104 worn on the head 154 of the user. FIG. 1C is another schematic view of the cochlear implant system 102, while FIG. 1D illustrates further details of the cochlear implant system 102. For ease of description, FIGS. 1A-1E will generally be described together.

Cochlear implant system 102 includes an external component 104 that is configured to be directly or indirectly attached to the body of the user and an implantable component 112 configured to be implanted in the user. In the examples of FIGS. 1A-1E, the external component 104 comprises a sound processing unit 106, while the cochlear implant 112 includes an implantable coil 114, an implant body 134, and an elongate stimulating assembly 116 configured to be implanted in the user's cochlea.

In the example of FIGS. 1A-1E, the sound processing unit 106 is an off-the-ear (OTE) sound processing unit, sometimes referred to herein as an OTE component, which is configured to send data and power to the implantable component 112. In general, an OTE sound processing unit is a component having a generally cylindrically shaped housing 111 and which is configured to be magnetically coupled to the user's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that can operate with implantable component 112. For example, in alternative examples, the external component can comprise a behind-the-ear (BTE) sound processing unit or a micro-BTE sound processing unit and a separate external. In general, a BTE sound processing unit comprises a housing that is shaped to be worn on the outer ear of the user and is connected to the separate external coil assembly via a cable, where the external coil assembly is configured to be magnetically and inductively coupled to the implantable coil 114. It is also to be appreciated that alternative external components can be located in the user's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the user. For example, the cochlear implant 112 can operate in a first general mode, sometimes referred to as an “external hearing mode,” in which the sound processing unit 106 captures sound signals which are then used as the basis for delivering stimulation signals to the user. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the user. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 can also operate in alternative modes.

In FIGS. 1A and 1C, the cochlear implant system 102 is shown with an external computing device 110, configured to implement aspects of the techniques presented. The computing device 110, which is shown in greater detail in FIG. 1E, is, for example, a personal computer, server computer, hand-held device, laptop device, multiprocessor system, microprocessor-based system, programmable consumer electronic (e.g., smart phone), network PC, minicomputer, mainframe computer, tablet, remote control unit, distributed computing environment that include any of the above systems or devices, and the like. The computing device 110 can be a single virtual or physical device operating in a networked environment over communication links to one or more remote devices, such as an implantable medical device or implantable medical device system.

In its most basic configuration, computing device 110 includes at least one processing unit 183 and memory 184. The processing unit 183 includes one or more hardware or software processors (e.g., Central Processing Units) that can obtain and execute instructions. The processing unit 183 can communicate with and control the performance of other components of the computing system 110.

The memory 184 is one or more software or hardware-based computer-readable storage media operable to store information accessible by the processing unit 183. The memory 184 can store, among other things, instructions executable by the processing unit 183 to implement applications or cause performance of operations described herein, as well as other data. The memory 184 can be volatile memory (e.g., RAM), non-volatile memory (e.g., ROM), or combinations thereof. The memory 884 can include transitory memory or non-transitory memory. The memory 184 can also include one or more removable or non-removable storage devices. In examples, the memory 184 can include RAM, ROM, EEPROM (Electronically-Erasable Programmable Read-Only Memory), flash memory, optical disc storage, magnetic storage, solid state storage, or any other memory media usable to store information for later access. In examples, the memory 184 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 184 can include wired media such as a wired network or direct-wired connection, and wireless media such as acoustic, RF, infrared and other wireless media or combinations thereof. In certain embodiments, the memory 184 comprises environmental sound training logic 185 that, when executed, enables the processing unit 183 to perform aspects of the techniques presented.

In the illustrated example, the system 110 further includes a network adapter 186, one or more input devices 187, and one or more output devices 188. The system 110 can include other components, such as a system bus, component interfaces, a graphics system, a power source (e.g., a battery), among other components.

The network adapter 186 is a component of the computing system 110 that provides network access (e.g., access to at least one network 189). The network adapter 186 can provide wired or wireless network access and can support one or more of a variety of communication technologies and protocols, such as ETHERNET, cellular, BLUETOOTH, near-field communication, and RF (Radiofrequency), among others. The network adapter 186 can include one or more antennas and associated components configured for wireless communication according to one or more wireless communication technologies and protocols.

The one or more input devices 187 are devices over which the computing system 110 receives input from a user. The one or more input devices 187 can include physically-actuatable user-interface elements (e.g., buttons, switches, or dials), touch screens, keyboards, mice, pens, and voice input devices, among others input devices.

The one or more output devices 188 are devices by which the computing system 810 is able to provide output to a user. The output devices 188 can include, a display 190 and one or more speakers 191, among other output devices.

It is to be appreciated that the arrangement for computing system 110 shown in FIG. 1E is merely illustrative and that aspects of the techniques presented herein can be implemented at a number of different types of systems/devices. For example, the computing system 110 can be a laptop computer, tablet computer, mobile phone, surgical system, etc.

The OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external device 110). However, it is to be appreciated that one or more input devices can include additional types of input devices and/or less input devices (e.g., the wireless short range radio transceiver 120 and/or one or more auxiliary input devices 128 can be omitted).

The OTE sound processing unit 106 also comprises the external coil 108, a charging coil 121, a closely-coupled transmitter/receiver (RF transceiver) 122, sometimes referred to as or radio-frequency (RF) transceiver 122, at least one rechargeable battery 132, and an external sound processing module 124. The external sound processing module 124 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intra-cochlear stimulating assembly 116, all configured to be implanted under the skin/tissue (tissue) 115 of the user. The implant body 134 generally comprises a hermetically-sealed housing 138 in which at least one battery 125, RF interface circuitry 140, and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the user's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the user's cochlea.

Stimulating assembly 116 extends through an opening in the user's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, can be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a user (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the user.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114.

That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the user's cochlea. In this way, cochlear implant system 102 electrically stimulates the user's auditory nerve cells, bypassing absent or defective hair cells that normally transduce acoustic vibrations into neural activity, in a manner that causes the user to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the user's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a user (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the user's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 can operate differently in different embodiments. For example, in one alternative implementation of the external hearing mode, the cochlear implant 112 can use signals captured by the sound input devices 118 and the implantable sound sensors 160 in generating stimulation signals for delivery to the user.

“Environmental sounds,” which as used herein refer to non-speech and non-musical ambient sounds, are a key part of an individual's everyday experience of her surroundings. That is, environmental sounds are acoustic signals that are distinguished from speech/music, and which convey meaningful information about objects and events in the user's surroundings. Environmental sounds can include, for example, a doorbell, a dog barking, pots clanging, etc. These environmental sounds can vary for different ambient sound environments (e.g., a first set of environmental sounds can be present in an outdoor park, but a second set of environmental sounds can be present in a kitchen).

Environmental sounds carry meanings and contextual information, that together provide situational awareness to an individual. That is, environmental sounds provide an individual with information about what is happening around her, where it is happening, and how it is happening. The perception of environmental sounds, along with speech, is one of the most important ecological functions served by human hearing. It creates greater awareness of the immediate dynamic environment, helps to avoid danger, and contributes to an overall sense of well-being.

Most listening environments (ambient sound environments) contain a great variety of acoustic signals, but researchers have largely concentrated on speech and, to a lesser extent, music. As such, the ability of hearing device users, such as cochlear implant users, to perceive environmental sounds has received little attention, despite cochlear users having considerably reduced environmental sound perception. Although many cochlear implant users develop some environmental sound perception without active training, their performance remains substantially lower than that of normal-hearing listeners.

The present inventors have recognized the importance of environmental sounds in the rich soundscapes of everyday life for hearing device users. In fact, perception of environmental sounds (i.e., non-speech and non-musical sounds that convey information about specific objects and events) plays an important role in enabling a hearing device user to navigate successfully through her daily environment. Moreover, everyday listening situations are often complex and involve multiple sound sources. Thus, to selectively listen to and identify a signal of importance among many, a hearing device user needs to learn to segregate the sounds in the complex auditory scene and group them into meaningful auditory objects or streams. This is a complex task because sounds are interleaved and overlap in both temporal and frequency domains, and the human auditory system only has access to an amalgam of all sounds that arrive at the ear at the same time.

The challenges that the brain is facing is hearing the correct target sound, ignoring the ambient soundscape, correctly processing these sounds, and responding to it rapidly. Many different sounds can arrive at the ears around the same time. All their spectro-temporal features are processed by the auditory system. However, auditory perception relies not only on the peripheral level but also requires higher order cognitive processing, such as learning, attention, and memory, which happens in cortical areas. Hearing impairment can affect this process of object formation and thus, hearing-impaired listeners generally perform worse than normal-hearing listeners in complex listening scenarios. This is the case even when hearing aids or cochlear implants are used to make the signals audible.

Accordingly, presented herein are techniques that are specifically designed to improve a hearing device user's perception of environmental sounds in complex listening scenarios. As described further below, the training can be delivered via a computing device (e.g., smartphone), such as external device 110, and can be delivered across several training levels/phases. The various training phases are described in greater detail below. It is to be appreciated that the specific described training phases are merely illustrative and that, in certain circumstances, the techniques presented herein can be implemented with different training phases, different orders for the training phases, etc. It also to be appreciated that the techniques presented herein can, in certain circumstances be implemented with a subset of the described training phases. For ease of reference, the different training phases will be described with reference to external device 110 and cochlear implant system 102.

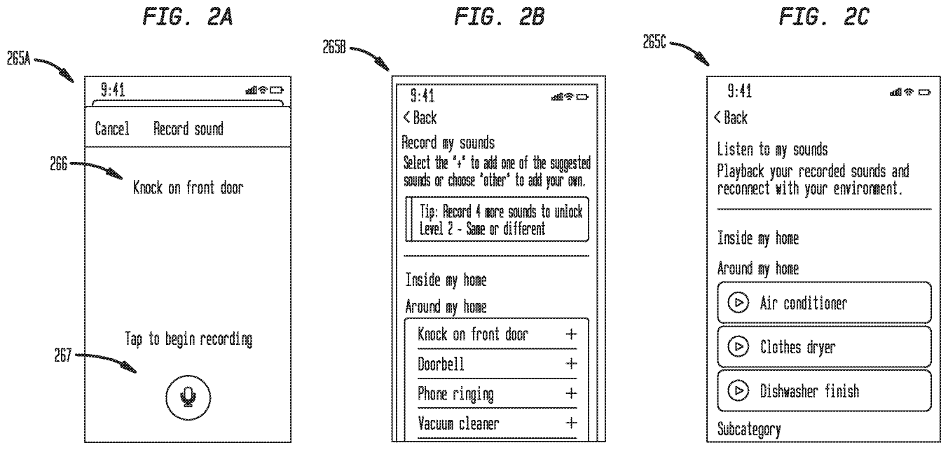

Referring first to FIGS. 2A, 2B, and 2C, shown are visual representations 265A, 265B, and 265C of example user interfaces that can be provided to a user in a first (initial) training phase (e.g., via a display 190), in accordance with certain embodiments presented herein. In this initial training phase, a hearing device user uses, for example, the computing device (e.g., smartphone) 110, cochlear implant system 102 (e.g., external component 104), and/or another device to record environmental sounds of most relevance to her daily life and the user is subsequently able to listen to these recorded environmental sounds (via the cochlear implant system 102). The listening to/playback of the recorded environmental sounds is sometimes referred to herein as initial “familiarization” training (e.g., a process during the user is able to become familiar with the environmental sounds, but is not tasked with discriminating between sounds or identifying sounds). In the initial familiarization training phase, the recorded environmental sounds are played in isolation (e.g., without any background noise).

The visual representation 265A of FIG. 2A represents a user interface that can be used by the user to actually record an environmental sound. In particular, shown in FIG. 2A is a first field 266 that allows the user to identify (name) the environmental sound, and an icon/button 267 that can be used to actually record the environmental sound. The visual representation 265B of FIG. 2B represents a user interface that can provide a suggested environmental sound checklist and/or environmental sound categories to guide the user to record the environmental sounds. Finally, the visual representation 265C of FIG. 2C represents a user interface that allows a user to playback the recorded environmental sounds (e.g., perform the familiarization training). As shown, once recorded, the environmental sounds can be added to the user's so called “environmental sound library.” The environmental sound library can be organized into categories and sub-categories that represent, for example, different ambient environments (e.g., categories can include inside home, car, office, while sub-categories can include kitchen, bush walk, etc.).

As noted, the user is able to listen to the recorded environmental sounds or, stated differently, the environmental sounds are provided to the user via a medical device, such as cochlear implant system 102. As used herein, the providing of environmental sounds to a user/listening to environmental sounds means that the medical device (e.g., cochlear implant system 102) delivers, to the user, one or more stimulation signals that represent the environmental sounds. For example, in certain embodiments, the recorded environmental sounds can be played via the one or more speakers 191 of the computing device 110, provided via a wireless connection from the computing device 110 to the cochlear implant system 102, etc. Thereafter, the sounds are processed by the cochlear implant system 102 and converted into stimulation signals that are delivered to the patient.

In certain embodiments, the user can select the preferred mode of delivery of the environmental sounds. For example, the user can, in one embodiment, select between (i) acoustic speaker output from the external device 110 and (ii) wireless streaming of the audio signal from the external device 110 to the cochlear implant system 102.

For ease of description, the techniques presented herein will be described with reference to delivery of environmental sound signals to a user, rather than with reference to delivery of stimulation signals representing the environmental sound signals to a user. Again, this nomenclature is merely for ease of description.

Also, for ease of illustration, the techniques presented herein will be described with reference to delivery of environmental sound signals to a user of a medical device (e.g., a user of a hearing device, namely cochlear implant system 102). However, it is also to be appreciated that the techniques presented herein can be implemented by a user that does not have or use any kind of hearing device (or other medical device).

The stimulation signals delivered to the user can vary depending on the type of medical device. For example, in the context of hearing devices, the stimulation signals representing the environmental sounds can be acoustic stimulation signals, mechanical stimulation signals, electrical stimulation signals, etc.

The initial familiarization training phase is generally implemented for a period of time so that the user can listen back to their environmental sound library at will for familiarization purposes. The user can generally select a specific sound to be played, thus providing the user with the knowledge of which sound she is hearing at a given time. The period of time provided for the user to familiarize herself with the environmental sounds can vary for different users. For example, in certain embodiments, progress is user-directed (e.g., the user can decide when she is ready to move to a next training phase based on feedback from the application).

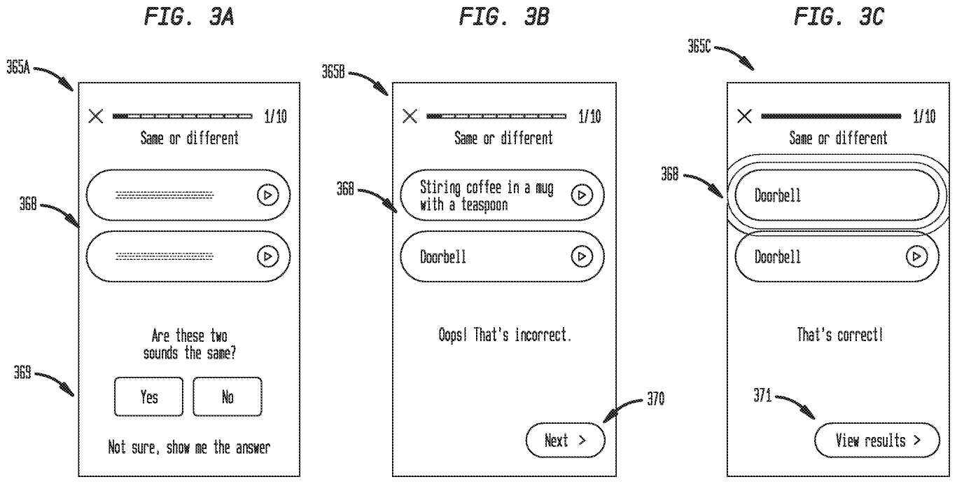

After the initial familiarization training phase, the techniques presented herein can initiate a discrimination training phase that provides training activities utilizing the user's custom recorded environmental sound library. More specifically, shown in FIGS. 3A, 3B, and 3C are visual representations 365A, 365B, and 365C, respectively, of user interfaces that can be provided to a user (e.g., via a display 190) to perform discrimination training.

Referring first to FIG. 3A, the visual representation 365A includes icons/buttons 368 that enable the user to play two recorded environmental sounds from her environmental sound library. As shown, the sounds that are played via icons 368 are not identified to the user (e.g., the sound names are omitted or obscured) and the sounds can be the same sound played twice or two different sounds. Visual representation 365A also includes icons 369 asking the user to indicate whether the two played sounds are the same sound (or different sounds). In the specific example of FIG. FIG. 3A, the icons 369 are “Yes” and “No” icons accompanied with text asking “Are these two sounds the same. That is, the user is asked to “discriminate” between the two sounds. The icons 369 can also be accompanied by a “Not sure, show me the answer” option that displays the names of the sounds to the user.

The visual representation 365B of FIG. 3B represents a user interface displayed if the user selects a wrong answer via icons 369. For example, in this specific illustration, the two sounds were “stirring coffee in a mug with a teaspoon” and “doorbell.” Within visual representation 365B, the name of the sounds within icons 368 are no longer obscured so that the user is made aware of what sounds she heard, and are accompanied by text indicating the user made the wrong selection at icons 369. The icons 368 can be activated by the user so that she can re-listen to each of the sounds one or more times. Finally, visual representation 365B also includes a continue/advance/next icon 370.

FIG. 3C includes a visual representation 365C that represents the alternative to FIG. 3B, namely a user interface displayed if the user selects the correct answer via icons 369 of FIG. 3A. For example, in this specific illustration, the two sounds were both “doorbell.” Within visual representation 365C, the name of the sounds within icons 368 are no longer obscured so that the user is made aware of what sounds she heard, and are accompanied by text indicating the user made the correct selection at icons 369. The icons 368 can be activated by the user so that she can re-listen to each of the sounds one or more times. Finally, visual representation 365B also includes a view results icon 370.

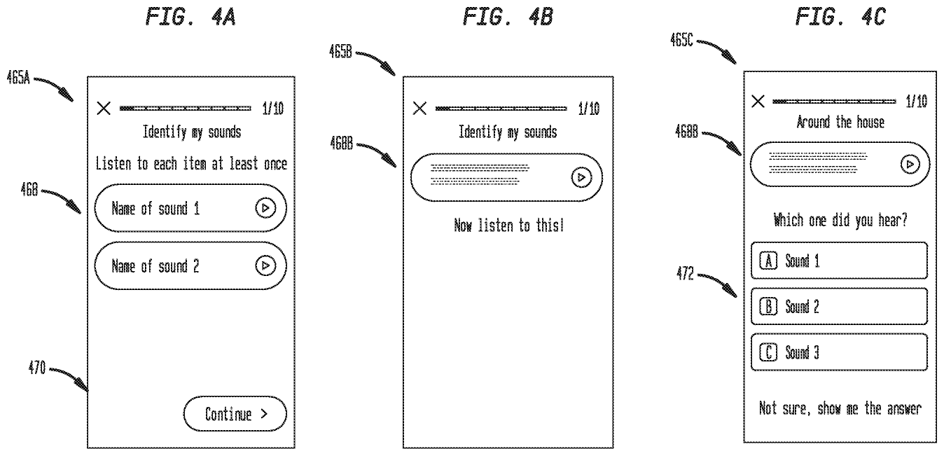

As noted, FIGS. 3A, 3B, and 3C illustrate discrimination training using the user's custom recorded environmental sound library. FIGS. 4A, 4B, and 4C include visual representations 465A, 465B, and 465C representing user interfaces that can be provided to a user to perform identification training using the user's custom recorded environmental sound library, in accordance with certain embodiments presented herein.

Referring first to FIG. 4A, the visual representation 465A includes icons/buttons 468 that enable the user to play two recorded environmental sounds from her environmental sound library. As shown, the sounds that are played via icons 468 are identified to the user (e.g., the sound names are displayed to the user). The icons 468 are accompanied with text instructing the user to listen to each of the two identified sounds at least one. After listening to these sounds, the user can select the continue/advance/next icon 470 to move to another user interface, represented by visual representation 465B.

The user interface represented by visual representation 465B includes an icon/button 468B that enables the user to play one of the two recorded environmental sounds that were played to the user from the user interface represented by visual representation 465A. However, within icon 468B, the associated sound is not identified to the user (e.g., the sound name is omitted or obscured). In the specific example of FIG. 4B, the icon 468B is accompanied with text instructing the user to listen to the sound (e.g., instructions to activate icon 468B, such as “Now listen to this!”). Activation of icons 468B can advance the process to a next user interface, represented by visual representation 465C.

As shown, visual representation 465C icon/button 468B, as well as icon/buttons 472 asking the user to indicate what sound she heard upon activation of the icon 468. In the example of FIG. 4C, the user is provided with several options (e.g., a multiple choice display) in order to provide the indication of what sound she heard. The use of a multiple choice display is merely illustrative and other forms for receiving the user's selection can be provided in alternative embodiments. For example, in other embodiments, the user can be provided with a box to type a response, the system can receive a verbal input, etc. The icons 472 can also be accompanied by a “Not sure, show me the answer” option that displays the names of the sounds to the user.

If the user selects a correct choice via icons 472, the user can be provided with a user interface that is similar to that of FIG. 3B. Conversely, if user selects an incorrect choice via icons 472, the user can be provided with a user interface that is similar to that of FIG. 3C.

The initial discrimination training phase (FIGS. 3A-3C) and the initial identification training phase (FIGS. 4A-4C) are each generally implemented for a period of time. The period of time for each of these training phases can vary for different users (e.g., the user can decide when she is ready to move to a next training phase based on feedback from the application).

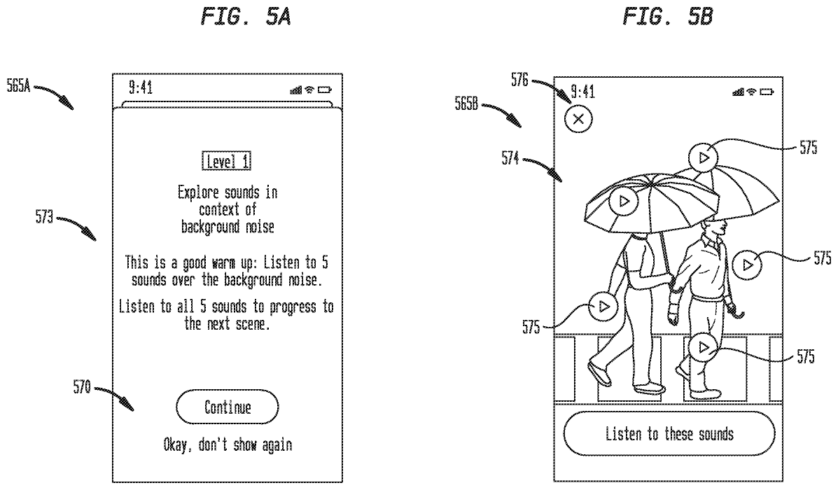

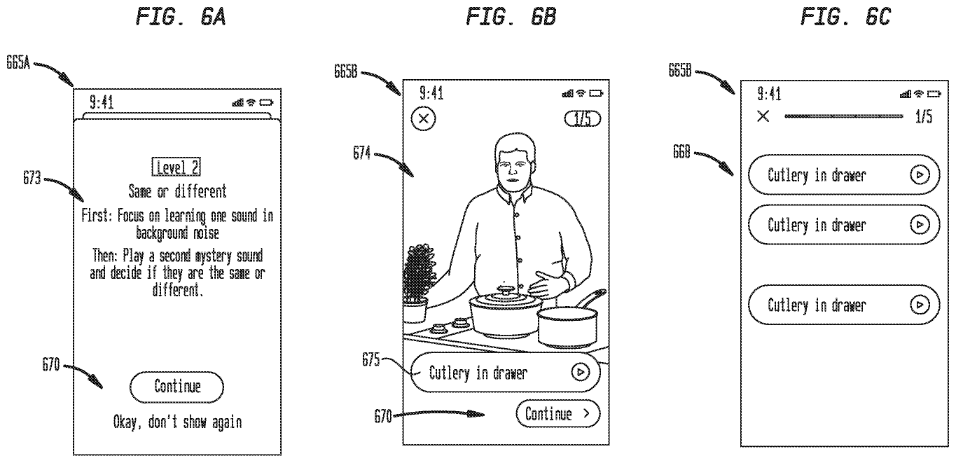

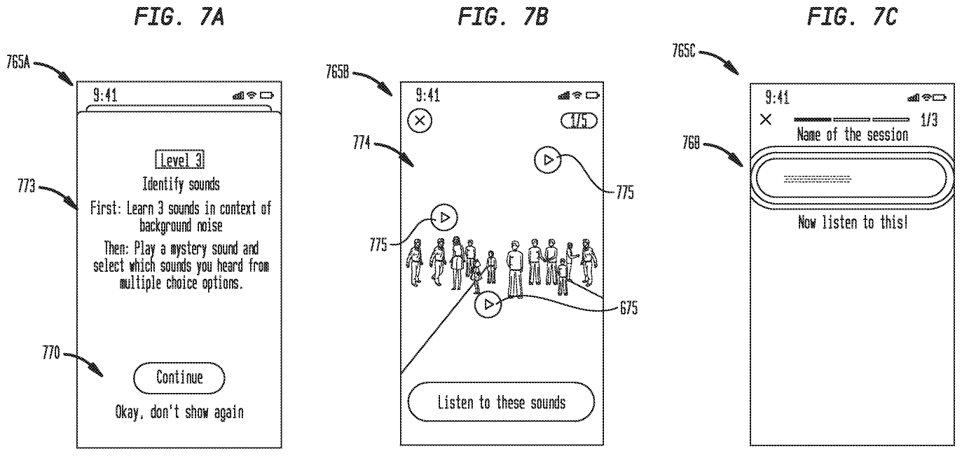

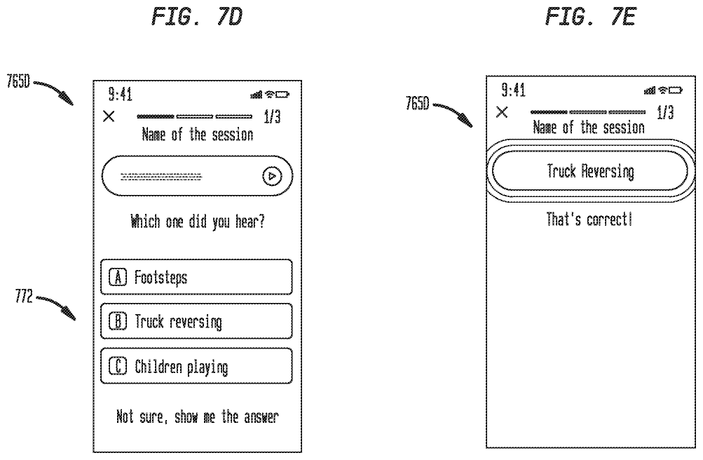

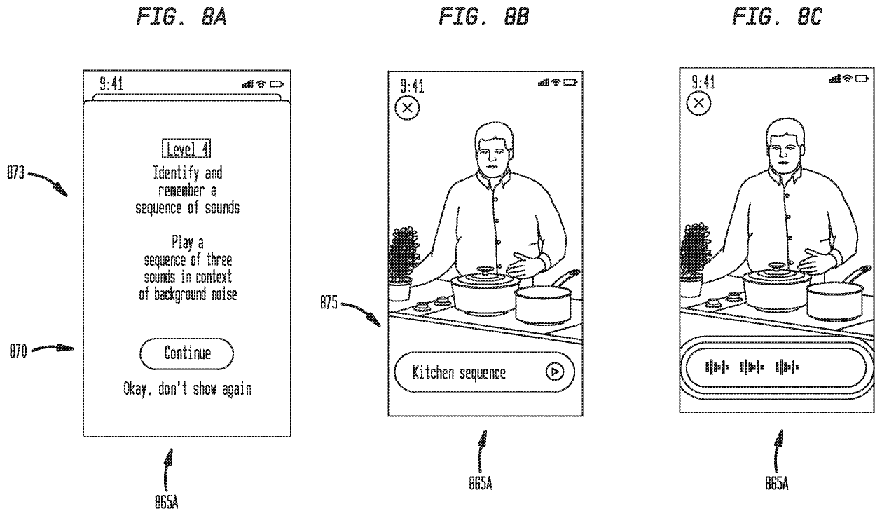

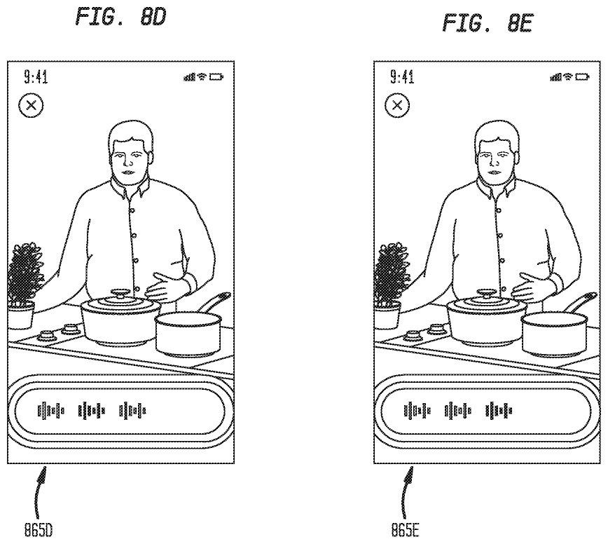

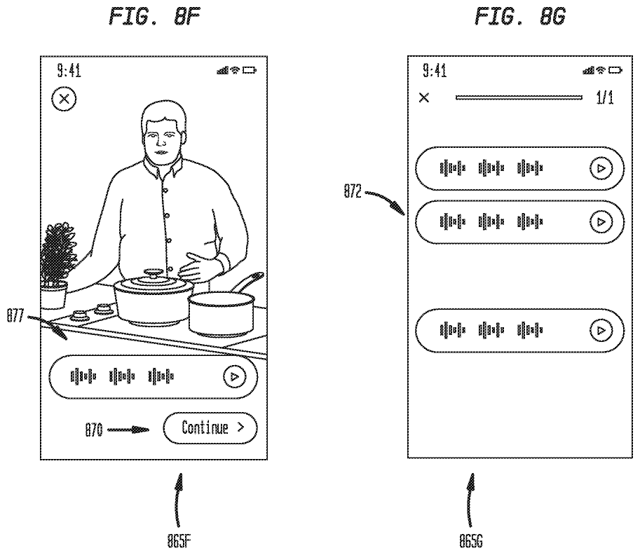

After the initial discrimination training phase and the initial identification training phase, the techniques presented herein can initiate a number of “advanced” training phases that provide training activities independent from the user's custom recorded environmental sound library (e.g., using pre-recorded sounds). In general, these advanced phases use thematic audio-visual soundscapes consisting of visual displays (e.g., cinemagraphs) with a closed set of target sounds embedded in ambient background sound (e.g., at +15 dB SNR). The visual displays, target sounds, and ambient auditory background are based on the soundscapes theme (e.g., supermarket, café, rainy street, park, kitchen, etc.). Described below are four example advanced training phases, referred to as an advanced familiarization training phase (FIGS. 5A and 5B), an advanced discrimination training phase (FIGS. 6A-6C), an advanced identification training phase (FIGS. 7A-7E), and a sequencing phase (FIGS. 8A-8G).

Referring first to FIGS. 5A and 5B, shown are visual representations 565A and 565B, respectively, of example user interfaces that can be provided to a user in an example advanced familiarization phase (e.g., via a display 190), in accordance with certain embodiments presented herein. In these examples, visual representation 565A illustrates an example instructions interface that provides instructions 573 explaining the advanced familiarization phase to the user. As shown in FIG. 5A, after reading the instructions 573, the user can select the continue/advance/next icon 570 to move to the user interface represented by visual representation 565B. The user can also have the option to skip or not display the instructions interface in the future.

The visual representation 565B illustrates an example interface to perform the advanced familiarization phase. As shown, the user interface represented by visual representation 565B includes a visual display 574 of an ambient sound environment or environmental sound scene (e.g., a visual display representing a specific ambient environment/soundscape). In the example of FIG. 5B, the ambient sound environment is an exploration phase of a sound walking experience, such as “exploring a rainy city street” and the visual display 574 is in the form of a cinema-graphic (animated photo image). The cinema-graphic image of FIG. 5B can include, for example, a visual representation of raining falling, blinking lights, etc.

It is to be appreciated that this specific example ambient sound environment, as well as the form of the visual display 574, are merely illustrative. In alternative embodiments, the visual display 574 can be, for example, a still photo image, a video display, an animated display, etc. Of course, any of a large number of ambient sound environments can also be envisioned for use with the techniques presented herein.

Returning to the example of FIG. 5B, the visual display 574 is overlaid with a plurality of icons/buttons 575 that allow the user to audibly explore the ambient sound environment via pre-recorded sound tokens. That is, each icon 575 is associated with a different environmental sound that would be found in the ambient sound environment represented by the visual display 574. Selecting/activating each of the icons 575 allows the user to listen to a different one of these sounds in the presence of background noise (e.g., closed set of target sounds embedded in ambient background sound where the target sounds are based on the soundscape theme). The environmental sounds are presented in ambient background noise to improve ecological validity.

As noted, in the embodiment of FIG. 5B, the ambient sound environment is a rainy city street and the sounds associated with the icons 575 can include, for example, a car horn, the sound of thunder, the sound of feet walking on wet pavement, etc. In general, the icons 575 provide interaction points with related sounds that the user would be expected to hear in this environment. The background noise can be, for example, the general sound of rain failing. The visual display 574 provides visual contextual cues for the environmental sounds so that the user “learns” how to interpret the sounds in the given ambient environment.

During the advanced familiarization phase, the user can be provided with a number of different ambient sound environments to audibly explore. In certain embodiments, the user is tasked with listening to all of the sounds (via icons 575) before she is permitted to explore another ambient sound environment. The user interface represented by the visual representation 565B includes, in this example, an icon 576 that enables the user to close/exit the user interface.

Referring next to FIGS. 6A-6C, shown are visual representations 665A, 665B, and 665C, respectively, of example user interfaces that can be provided to a user in an example advanced discrimination training phase (e.g., via a display 190), in accordance with certain embodiments presented herein. In these examples, visual representation 665A illustrates an example instructions interface that provides instructions 673 explaining the advanced discrimination training to the user. As shown in FIG. 6A, after reading the instructions 673, the user can select the continue/advance/next icon 670 to move to the user interface represented by visual representation 665B. The user can also have the option to skip or not display the instructions interface in the future.

The visual representation 665B illustrates an example interface to perform a first part of the advanced discrimination training phase. As shown, the user interface represented by visual representation 665B includes a visual display 674 of an ambient sound environment or environmental sound scene (e.g., a visual display representing a specific ambient environment/soundscape). In the example of FIG. 6B, the ambient sound environment is a “kitchen” and the visual display 674 is in the form of a cinema-graphic (animated photo image). The cinema-graphic image of FIG. 6B can include, for example, a visual representation of a stove flame, steam rising, etc.

As noted above, the specific example ambient sound environment of FIG. 6B, as well as the form of the visual display 674, are merely illustrative and, in alternative embodiments, the visual display 674 can be, for example, a still photo image, a video display, an animated display, etc. In addition, the kitchen environment of FIG. 6B is just one of a large number of ambient sound environments can be used with the techniques presented herein.

Returning to the example of FIG. 6B, the visual display 674 is overlaid with an icons/button 675 that allows the user to play a selected/target environmental sound associated with the ambient sound environment represented by the visual display 674. That is, selecting/activating the icon 675 allows the user to listen to a selected “kitchen” sound in the presence of background noise. In the embodiment of FIG. 6B, the selected kitchen sound is “cutlery in a drawer,” but again this is merely illustrative. Other target sounds can include, for example, the sound of steam rising, the sound of pots clanging, etc. The visual display 674 provides visual contextual cues for the target environmental sound. Moreover, the user interface represented by visual representation 665B identifies the target environmental sound to the user (e.g., the icon 675 includes, or is accompanied by, text indicating “cutlery in a drawer”).

After the user listens to the target environmental sound via icon 675, she can select the continue/advance/next icon 670 to move to the user interface represented by visual representation 665C. The user interface represented by visual representation 665C tasks the user to perform a discrimination test between the target environmental sound provided in the user interface represented by visual representation 665B (i.e., the sound played via selection of icon 675) and a pre-recorded unidentified/mystery target environmental sound, which is also associated with the ambient sound environment represented by visual display 674. The mystery sound can be the target environmental sound, or a different environmental sound. To this end, the user interface represented by visual representation 665C includes icons/buttons 668 that enable the user to play each of the target environmental sound and the unidentified sound.

After the user plays both environmental sounds, the user is asked, in some form, to indicate whether the two played sounds are the same sound (or different sounds). That is, the user is asked to “discriminate” between the two environmental sounds. For example, visual representation 665C can include “Yes” and “No” icons accompanied with text asking “Are these two sounds the same. The icons can also be accompanied by a “Not sure, show me the answer” option that displays the names of the sounds to the user.

If the user provides a correct response, the techniques can, for example, return to the user interface represented by FIG. 6B and a repeat of the above with a different target environmental sound and/or move to another advanced discrimination test similar to the above with a different ambient sound environment.

However, an incorrect response initiates an intervention process. In this intervention process, the system will again play the target environmental sound with the visual context (e.g., play the sound while providing the visual display 674). Thereafter, the system will present the target environmental sound in quiet (e.g., no ambient background noise and no visual display) and then present the target environmental sound with noise/ambient background (and no visual display). After the intervention, the techniques can, for example, return to the user interface represented by FIG. 6B and a repeat of the above with a different target environmental sound and/or move to another advanced discrimination test similar to the above with a different ambient sound environment.

During the advanced discrimination training phase, the user can be provided with discrimination training using a number of different ambient sound environments with different environmental sounds.

Referring next to FIGS. 7A-7E, shown are visual representations 765A, 765B, 765C, 765D, and 765E respectively, of example user interfaces that can be provided to a user in an example advanced identification training phase (e.g., via a display 190), in accordance with certain embodiments presented herein. In these examples, visual representation 765A illustrates an example instructions interface that provides instructions 773 explaining the advanced identification training to the user. As shown in FIG. 7A, after reading the instructions 773, the user can select the continue/advance/next icon 770 to move to the user interface represented by visual representation 765B. The user can also have the option to skip or not display the instructions interface in the future.

The visual representation 765B illustrates an example interface to perform a first part of the advanced identification training phase. As shown, the user interface represented by visual representation 765B includes a visual display 774 of an ambient sound environment or environmental sound scene (e.g., a visual display representing a specific ambient environment/soundscape). In the example of FIG. 7B, the ambient sound environment is an “outdoor park” and the visual display 774 is in the form of a cinema-graphic (animated photo image). The cinema-graphic image of FIG. 7B can include, for example, a visual representation of falling leaves, moving branches, etc.

As noted above, the specific example ambient sound environment of FIG. 7B, as well as the form of the visual display 774, are merely illustrative and, in alternative embodiments, the visual display 774 can be, for example, a still photo image, a video display, an animated display, etc. In addition, the park environment of FIG. 7B is just one of a large number of ambient sound environments can be used with the techniques presented herein.

Returning to the example of FIG. 7B, the visual display 774 is overlaid with a plurality of icons/buttons 775 that allow the user to audibly explore the ambient sound environment via pre-recorded sound tokens. That is, each icon 775 is associated with a different environmental sound that would be found in the ambient sound environment represented by the visual display 774. Selecting/activating each of the icons 775 allows the user to listen to a different one of these sounds in the presence of background noise. The sounds are presented in ambient background noise to improve ecological validity.

As noted, in the embodiment of FIG. 7B, the ambient sound environment is the exploration of an outdoor park and the sounds associated with the icons 775 can include, for example, the sound of children playing, the sound of a sports whistle, the sound of footsteps, the sound of a truck reversing, etc. In general, the icons 775 provide interaction points with related sounds that the user would be expected to hear in this ambient sound environment. The visual display 774 provides visual contextual cues for the environmental sounds so that the user “learns” how to interpret the sounds in the given ambient environment.

After listening to these sounds via the user interface represented by visual represent 765B, the user can move to another user interface, represented by visual representation 765C, which includes an icon/button 768 that enables the user to play a selected one of the environmental sounds associated with visual display 774 (e.g., one of the environmental sounds that were played to the user from the user interface represented by visual representation 765B). The selected one of the environmental sounds associated with visual display 774 is sometimes referred to herein as the target environmental sound. However, within icon 768, the associated target sound is not identified to the user (e.g., the environmental sound name is omitted or obscured). In the specific example of FIG. 7C, the icon 768 is accompanied with text instructing the user to listen to the sound (e.g., instructions to activate icon 768, such as “Now listen to this!”). Activation of icon 768 can advance the process to a next user interface, represented by visual representation 765D.

As shown, visual representation 765D includes the icon 768, as well as icon/buttons 772 asking the user to indicate what sound she heard upon activation of the icon 768. In the example of FIG. 7D, the user is provided with several options (e.g., a multiple choice display) in order to provide the indication of what sound she heard. The use of a multiple choice display is merely illustrative and other forms for receiving the user's selection can be provided in alternative embodiments. For example, in other embodiments, the user can be provided with a box to type a response, the system can receive a verbal input, etc. The icons 772 can also be accompanied by a “Not sure, show me the answer” option that displays the names of the sounds to the user.

If the user selects a correct choice via icons 772, the user can be provided with a user interface, represented by FIG. 7E. Thereafter, the techniques can, for example, return to the user interface represented by FIG. 7B and a repeat of the above with a different target environmental sound and/or move to another advanced identification test similar to the above with a different ambient sound environment.

However, an incorrect response initiates an intervention process. In this intervention process, the system will again play the target environmental sound with the visual context (e.g., play the sound while providing the visual display 774). Thereafter, the system will present the target environmental sound in quiet (e.g., no ambient background noise and no visual display) and then present the target environmental sound with noise/ambient background (and no visual display). After the intervention, the techniques can, for example, return to the user interface represented by FIG. 7B and a repeat of the above with a different target environmental sound and/or move to another advanced discrimination test similar to the above with a different ambient sound environment.

During the advanced identification training phase, the user can be provided with identification training using a number of different ambient sound environments with different environmental sounds.

Turning next to FIGS. 8A-8G, shown are a series of visual representations 865A-865G, respectively, of example user interfaces that can be provided to a user in an example sound sequencing phase (e.g., via a display 190), in accordance with certain embodiments presented herein. In these examples, visual representation 865A illustrates an example instructions interface that provides instructions 873 explaining the sound sequencing training o the user. As shown in FIG. 8A, after reading the instructions 873, the user can select the continue/advance/next icon 870 to move to the user interface represented by visual representation 865B. The user can also have the option to skip or not display the instructions interface in the future.

The visual representation 865B illustrates an example interface to perform a first part of the sound sequencing training phase. As shown, the user interface represented by visual representation 865B includes a visual display 874 of an ambient sound environment or environmental sound scene (e.g., a visual display representing a specific ambient environment/soundscape). In the example of FIG. 8B, the ambient sound environment is a “kitchen” and the visual display 874 is in the form of a cinema-graphic (animated photo image). The cinema-graphic image of FIG. 8B can include, for example, a visual representation of a stove flame, steam rising, etc.

As noted above, the specific example ambient sound environment of FIG. 8B, as well as the form of the visual display 874, are merely illustrative and, in alternative embodiments, the visual display 874 can be, for example, a still photo image, a video display, an animated display, etc. In addition, the kitchen environment of FIG. 8B is just one of a large number of ambient sound environments can be used with the techniques presented herein.

Returning to the example of FIG. 8B, the visual display 674 is overlaid with an icons/button 875 that allows the user to play a selected/target sequences of environmental sounds associated with the ambient sound environment represented by the visual display 874. That is, selecting/activating the icon 875 allows the user to listen to a selected “kitchen sound sequence” comprising three (3) sounds in the presence of background noise (e.g., knife chopping, kettle whistling, pots clanging). FIGS. 8C, 8D, and 8E are visual representations 865C, 865D, and 865E that illustrate the playing of the sequence of the three selected kitchen sounds to the user. FIG. 8F is a visual representation 865F of a user interface with a button/icon 877 that allows the user to re-listen to the sequence of sounds, as well as another continue icon 870.

Visual representation 865G of FIG. 8G represents a user interface that can be provided to the user after she listens to the sound sequence one or more times. The user interface represented by visual representation 865G includes well as icon/buttons 872 asking the user to select the correct sequence/order of the sounds she heard upon activation of the icon 875 (FIG. 8B). In the example of FIG. 8G, the user is provided with several options (e.g., a multiple choice display) in order to provide the indication of what sound order/sequence she heard. The use of a multiple choice display is merely illustrative and other forms for receiving the user's selection can be provided in alternative embodiments.

During the sound sequencing training phase, the user can be provided with identification training using a number of different ambient sound environments with different environmental sounds. In general, the sound sequencing training places increasing demands on the user's working memory by focusing on the ability to recall the order of sound sequences (e.g., sequencing is a process within working memory).

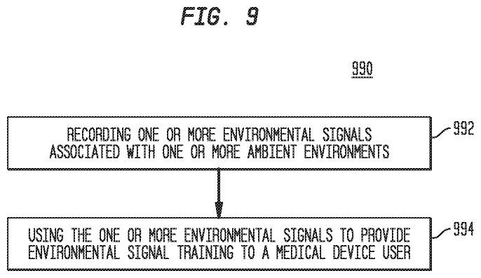

FIG. 9 is a flowchart of an example method 990, in accordance with embodiments presented herein. Method 990 begins at 992 where one or more environmental signals associated with one or more ambient environments are recorded at a computing device. At 994, the one or more environmental signals recorded at the computing device are used to provide environmental signal training to a medical device (e.g., hearing device) user.

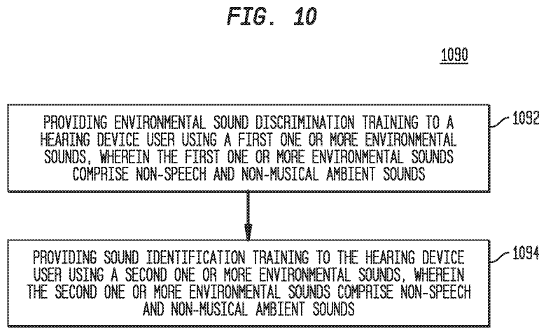

FIG. 10 is a flowchart of an example method 1090, in accordance with embodiments presented herein. Method 1090 begins at 1092 where environmental sound discrimination training is provided to a hearing device user using a first one or more environmental sounds, wherein the first one or more environmental sounds comprise non-speech and non-musical ambient sounds. At 1094, environmental sound identification training is provided to the hearing device user using a second one or more environmental sounds, wherein the second one or more environmental sounds comprise non-speech and non-musical ambient sounds.

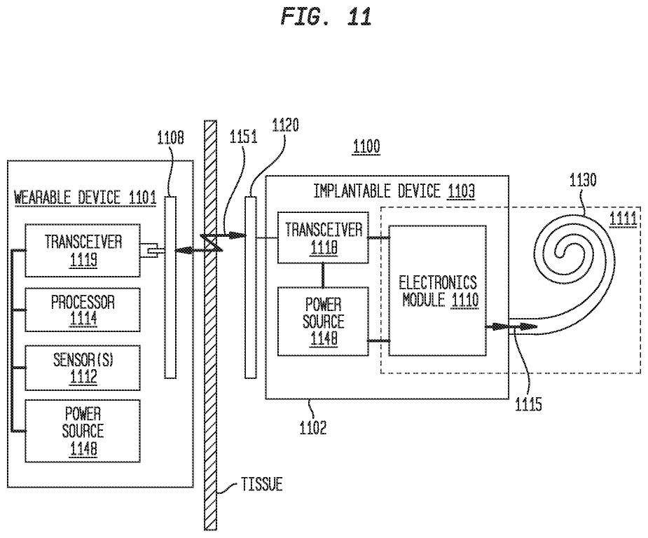

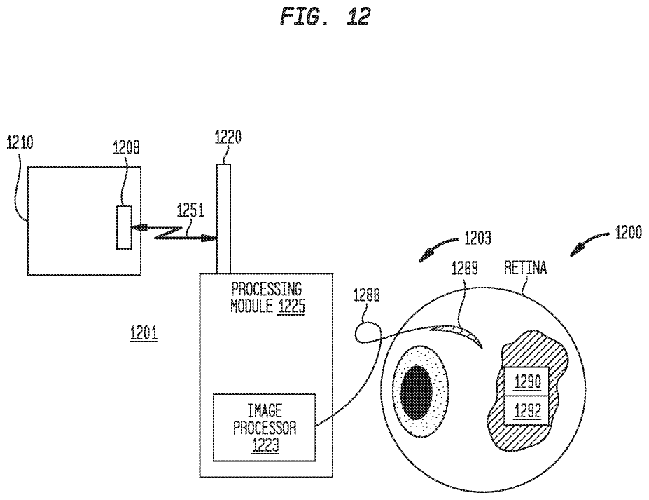

As previously described, the technology disclosed herein can be applied in any of a variety of circumstances and with a variety of different devices. Example devices that can benefit from technology disclosed herein are described in more detail in FIGS. 11 and 12, below. For example, the techniques described herein can be implemented through wearable medical devices, such as an implantable stimulation system as described in FIG. 11, or a retinal prosthesis as described in FIG. 12. The techniques of the present disclosure can be applied to other medical devices, such as neurostimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 11 is a functional block diagram of an implantable stimulator system 1100 that can benefit from the technologies described herein. The implantable stimulator system 1100 includes the wearable device 1101 acting as an external processor device and an implantable device 1103 acting as an implanted stimulator device. In examples, the implantable device 1103 is an implantable stimulator device configured to be implanted beneath a recipient's tissue (e.g., skin). In examples, the implantable device 1103 includes a biocompatible implantable housing 1102. Here, the wearable device 1101 is configured to transcutaneously couple with the implantable device 1103 via a wireless connection to provide additional functionality to the implantable device 30.

In the illustrated example, the wearable device 1101 includes one or more sensors 1112, a processor 1114, a transceiver 1118, and a power source 1148. The one or more sensors 1112 can be one or more units configured to produce data based on sensed activities. In an example where the stimulation system 1100 is an auditory prosthesis system, the one or more sensors 1112 include sound input sensors, such as a microphone, an electrical input for an FM hearing system, other components for receiving sound input, or combinations thereof. Where the stimulation system 1100 is a visual prosthesis system, the one or more sensors 1112 can include one or more cameras or other visual sensors. The processor 1114 can be a component (e.g., a central processing unit) configured to control stimulation provided by the implantable device 30. The stimulation can be controlled based on data from the sensor 1112, a stimulation schedule, or other data. Where the stimulation system 1100 is an auditory prosthesis, the processor 1114 can be configured to convert sound signals received from the sensor(s) 1112 (e.g., acting as a sound input unit) into signals 1151. The transceiver 1118 is configured to send the signals 1151 in the form of power signals, data signals, combinations thereof (e.g., by interleaving the signals), or other signals. The transceiver 1118 can also be configured to receive power or data. Stimulation signals can be generated by the processor 1114 and transmitted, using the transceiver 1118, to the implantable device 1103 for use in providing stimulation.

In the illustrated example, the implantable device 1103 includes a transceiver 1118, a power source 1148, and a medical instrument 1111 that includes an electronics module 1110 and a stimulator assembly 1130. The implantable device 1103 further includes a hermetically sealed, biocompatible implantable housing 1102 enclosing one or more of the components.

The electronics module 1110 can include one or more other components to provide medical device functionality. In many examples, the electronics module 1110 includes one or more components for receiving a signal and converting the signal into the stimulation signal 1115. The electronics module 1110 can further include a stimulator unit. The electronics module 1110 can generate or control delivery of the stimulation signals 1115 to the stimulator assembly 1130. In examples, the electronics module 1110 includes one or more processors (e.g., central processing units or microcontrollers) coupled to memory components (e.g., flash memory) storing instructions that when executed cause performance of an operation. In examples, the electronics module 1110 generates and monitors parameters associated with generating and delivering the stimulus (e.g., output voltage, output current, or line impedance). In examples, the electronics module 1110 generates a telemetry signal (e.g., a data signal) that includes telemetry data. The electronics module 1110 can send the telemetry signal to the wearable device 1101 or store the telemetry signal in memory for later use or retrieval.

The stimulator assembly 1130 can be a component configured to provide stimulation to target tissue. In the illustrated example, the stimulator assembly 1130 is an electrode assembly that includes an array of electrode contacts disposed on a lead. The lead can be disposed proximate tissue to be stimulated. Where the system 1100 is a cochlear implant system, the stimulator assembly 1130 can be inserted into the recipient's cochlea. The stimulator assembly 1130 can be configured to deliver stimulation signals 1115 (e.g., electrical stimulation signals) generated by the electronics module 1110 to the cochlea to cause the recipient to experience a hearing percept. In other examples, the stimulator assembly 1130 is a vibratory actuator disposed inside or outside of a housing of the implantable device 1103 and configured to generate vibrations. The vibratory actuator receives the stimulation signals 1115 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion.

The transceivers 1118 can be components configured to transcutaneously receive and/or transmit a signal 1151 (e.g., a power signal and/or a data signal). The transceiver 1118 can be a collection of one or more components that form part of a transcutaneous energy or data transfer system to transfer the signal 1151 between the wearable device 1101 and the implantable device 30. Various types of signal transfer, such as electromagnetic, capacitive, and inductive transfer, can be used to usably receive or transmit the signal 1151. The transceiver 1118 can include or be electrically connected to a coil 20.

As illustrated, the wearable device 1101 includes a coil 1108 for transcutaneous transfer of signals with coil 1120. As noted above, the transcutaneous transfer of signals between coil 1108 and the coil 1120 can include the transfer of power and/or data from the coil 1108 to the coil 1120 and/or the transfer of data from coil 1120 to the coil 1108. The power source 1148 can be one or more components configured to provide operational power to other components. The power source 1148 can be or include one or more rechargeable batteries. Power for the batteries can be received from a source and stored in the battery. The power can then be distributed to the other components as needed for operation.

As should be appreciated, while particular components are described in conjunction with FIG. 11, technology disclosed herein can be applied in any of a variety of circumstances. The above discussion is not meant to suggest that the disclosed techniques are only suitable for implementation within systems akin to that illustrated in and described with respect to FIG. 11. In general, additional configurations can be used to practice the methods and systems herein and/or some aspects described can be excluded without departing from the methods and systems disclosed herein.

FIG. 12 illustrates a retinal prosthesis system 1201 that comprises an external device 1210 (which can correspond to the wearable device 1101) configured to communicate with a retinal prosthesis 1200 via signals 1251. The retinal prosthesis 1200 comprises an implanted processing module 1225 (e.g., which can correspond to the implantable device 1103) and a retinal prosthesis sensor-stimulator 1290 is positioned proximate the retina of a recipient. The external device 1210 and the processing module 1225 can communicate via coils 1208, 1220.

In an example, sensory inputs (e.g., photons entering the eye) are absorbed by a microelectronic array of the sensor-stimulator 1290 that is hybridized to a glass piece 1292 including, for example, an embedded array of microwires. The glass can have a curved surface that conforms to the inner radius of the retina. The sensor-stimulator 1290 can include a microelectronic imaging device that can be made of thin silicon containing integrated circuitry that convert the incident photons to an electronic charge.

The processing module 1225 includes an image processor 1223 that is in signal communication with the sensor-stimulator 1290 via, for example, a lead 1288 which extends through surgical incision 1289 formed in the eye wall. In other examples, processing module 1225 is in wireless communication with the sensor-stimulator 1290. The image processor 1223 processes the input into the sensor-stimulator 1290, and provides control signals back to the sensor-stimulator 1290 so the device can provide an output to the optic nerve. That said, in an alternate example, the processing is executed by a component proximate to, or integrated with, the sensor-stimulator 1290. The electric charge resulting from the conversion of the incident photons is converted to a proportional amount of electronic current which is input to a nearby retinal cell layer. The cells fire and a signal is sent to the optic nerve, thus inducing a sight perception.

The processing module 1225 can be implanted in the recipient and function by communicating with the external device 1210, such as a behind-the-ear unit, a pair of eyeglasses, etc. The external device 1210 can include an external light/image capture device (e.g., located in/on a behind-the-ear device or a pair of glasses, etc.), while, as noted above, in some examples, the sensor-stimulator 1290 captures light/images, which sensor-stimulator is implanted in the recipient.

As noted above, presented herein are techniques for training a medical device (e.g., hearing device) user to correctly perceive environmental signals. Much of the above disclosure has been described with reference to environmental signal in the form of environmental sound signals. However, as noted above, the techniques presented herein can also be implemented with other types of environmental sound signals. For example, the techniques presented herein can be applied in the retinal prosthesis system 1201 to provide a user with training in relation to, for example, visual signals, such as colors, lighting, still images, video, etc. For example, the techniques presented herein can be implemented to provide the user with environmental signal discrimination training and/or environmental signal identification training on visual, rather than audible, environmental signals.

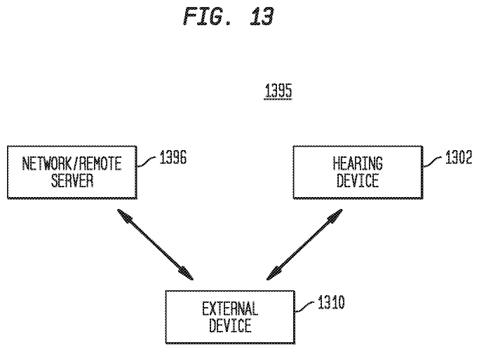

FIG. 13 is a functional block diagram of a computing environment 1395 with which aspects of the techniques presented herein can be implemented. As shown, the computing environment 1395 comprises a hearing device 1302 (e.g., implantable prosthesis, non-implantable prosthesis, or consumer hearing device such as earbuds, a bone conduction consumer hearing headset and the like), an external device 1310 (e.g., a phone, watch, tablet, or the like), and a network/remote server 1396. In operation, various aspects of the techniques can be implemented at any of the hearing device 1302, the external device 1310, and/or the network/remote server 1396.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. A method, comprising:
recording, at a computing device, one or more environmental signals associated with one or more ambient environments; and
using the one or more environmental signals recorded at the computing device to provide environmental signal training to a medical device user.

2. The method of claim 1, wherein recording the one or more environmental signals associated with the one or more ambient environments comprises:
recording one or more environmental sounds associated with one or more ambient sound environments.

3. The method of claim 2, wherein using the one or more environmental signals to provide environmental signal training to the medical device user comprises:
using the one or more environmental sounds to provide environmental sound training to a hearing device user.

4. The method of claim 3, using the one or more environmental sounds to provide environmental sound training to the hearing device user comprises:
using the one or more environmental sounds to provide environmental sound training to a cochlear implant user.

5. The method of claim 3, wherein using the one or more environmental signals to provide environmental sound training to the hearing device user comprises:
using the one or more environmental signals to provide environmental sound familiarization training to the hearing device user.

6. The method of claim 3, using the one or more environmental sounds to provide environmental sound training to the hearing device user comprises:
using the one or more environmental sounds to provide environmental sound discrimination training to the hearing device user.

7. The method of claim 6, wherein using the one or more environmental sounds to provide environmental sound discrimination training to the hearing device user comprises:
delivering at least one stimulation signal representing a first one of the one or more environmental sounds to the hearing device user;
delivering at least one stimulation signal representing a second one of the one or more environmental sounds to the hearing device user; and
providing, via a display screen, at least one input for use by the hearing device user to indicate whether the hearing device user perceived a difference between the at least one stimulation signal representing a first one of the one or more environmental sounds and the at least one stimulation signal representing a second one of the one or more environmental sounds.

8. The method of claim 3, wherein using the one or more environmental signals to provide environmental sound training to the hearing device user comprises:
using the one or more environmental signals to provide sound identification training to the hearing device user.

9. The method of claim 8, wherein using the one or more environmental sounds to provide sound identification training to the hearing device user comprises:
delivering at least one stimulation signal representing a first one of the one or more environmental sounds to a user; and
providing, via a display screen, at least one input for use by the user to identify the first one of the one or more environmental sounds.

10. The method of claim 3, further comprising:
providing, to the hearing device user via a display screen, a visual display representing an ambient sound environment; and
providing the hearing device user with environmental sound training using one or more environmental sounds associated with the ambient sound environment represented by the visual display.

11. The method of claim 10, wherein providing the hearing device user with environmental sound training using one or more environmental sounds associated with the ambient sound environment represented by the visual display comprises:
delivering, to the hearing device user along with the visual display, one or more stimulation signals representing at least one environmental sound associated with the ambient sound environment represented by the visual display.

12. The method of claim 10, wherein providing the hearing device user with environmental sound training using one or more environmental sounds associated with the ambient sound environment represented by the visual display comprises:
delivering at least one stimulation signal representing a first one of the one or more environmental sounds to the hearing device user;
delivering at least one stimulation signal representing a second one of the one or more environmental sounds to the hearing device user; and
providing, via the display screen, at least one input for use by the hearing device user to indicate whether the hearing device user perceived a difference between the at least one stimulation signal representing the first one of the one or more environmental sounds and the at least one stimulation signal representing the second one of the one or more environmental sounds.

13. The method of claim 10, wherein providing the hearing device user with environmental sound training using one or more environmental sounds associated with the ambient sound environment represented by the visual display comprises:
delivering at least one stimulation signal representing a first one of the one or more environmental sounds to the hearing device user; and
providing, via the display screen, at least one input for use by the hearing device user to identify the first one of the one or more environmental sounds.

14. The method of claim 10, wherein providing the hearing device user with environmental sound training using one or more environmental sounds associated with the ambient sound environment represented by the visual display comprises:
delivering a sequence of stimulation signals representing a plurality of environmental sounds to the hearing device user; and
providing, via the display screen, at least one input for use by the hearing device user to identify a sequence of the plurality of environmental sounds.

15. The method of claim 1, further comprising:
providing the medical device user with suggestions of environmental signals to record.

16-28. (canceled)

29. One or more non-transitory computer readable storage media comprising instructions that, when executed by a processor, cause the processor to:
deliver one or more user interfaces enabling a user to record one or more environmental sounds;
store the one or more environmental sounds in an environmental sound library; and
provide environmental sound training to a hearing device user using at least one of the one or more environmental sounds stored in the environmental sound library.

30. The one or more non-transitory computer readable storage media of claim 29, wherein the instructions operable to provide environmental sound training to the hearing device user using at least one of the one or more environmental sounds stored in the environmental sound library comprise instructions operable to:
provide environmental sound familiarization training to the hearing device user using at least one of the one or more environmental sounds stored in the environmental sound library.

31. The one or more non-transitory computer readable storage media of claim 29, wherein the instructions operable to provide environmental sound training to the hearing device user using at least one of the one or more environmental sounds stored in the environmental sound library comprise instructions operable to:
provide environmental sound discrimination training to the hearing device user using at least one of the one or more environmental sounds stored in the environmental sound library.

32. The one or more non-transitory computer readable storage media of claim 29, wherein the instructions operable to provide environmental sound training to the hearing device user using at least one of the one or more environmental sounds stored in the environmental sound library comprise instructions operable to:
provide environmental sound identification training to the hearing device user using at least one of the one or more environmental sounds stored in the environmental sound library.

33. The one or more non-transitory computer readable storage media of claim 29, further comprising instructions operable to:
provide, to the hearing device user via a display screen, a visual display representing an ambient sound environment; and
provide the hearing device user with environmental sound training using one or more pre-recorded environmental sounds associated with the ambient sound environment represented by the visual display.

34. The one or more non-transitory computer readable storage media of claim 33, wherein the instructions operable to provide the hearing device user with environmental sound training using one or more pre-recorded environmental sounds associated with the ambient sound environment represented by the visual display comprise instructions operable to:
while providing the visual display, provide at least one pre-recorded environmental sound associated with the ambient sound environment represented by the visual display.

35. The one or more non-transitory computer readable storage media of claim 33, wherein the instructions operable to provide the hearing device user with environmental sound training using one or more pre-recorded environmental sounds associated with the ambient sound environment represented by the visual display comprise instructions operable to:
while providing the visual display, provide a first one of the one or more pre-recorded environmental sounds to the hearing device user;
while providing the visual display, provide a second one of the one or more pre-recorded environmental sounds to the hearing device user; and
provide, via the display screen, at least one input for use by the hearing device user to indicate whether the user perceived a difference between the first one of the one or more pre-recorded environmental sounds and the second one of the one or more pre-recorded environmental sounds.

36. The one or more non-transitory computer readable storage media of claim 33, wherein the instructions operable to provide the hearing device user with environmental sound training using one or more pre-recorded environmental sounds associated with the ambient sound environment represented by the visual display comprise instructions operable to:
while providing the visual display, provide a first one of the one or more pre-recorded environmental sounds to the hearing device user; and
providing, via the display screen, at least one input for use by the hearing device user to identify the first one of the one or more pre-recorded environmental sounds.

37. The one or more non-transitory computer readable storage media of claim 33, wherein the instructions operable to provide the hearing device user with environmental sound training using one or more pre-recorded environmental sounds associated with the ambient sound environment represented by the visual display comprise instructions operable to:
while providing the visual display, provide a plurality of environmental sounds to the hearing device user; and
provide, via the display screen, at least one input for use by the hearing device user to identify a sequence of the plurality of environmental sounds.

38. The one or more non-transitory computer readable storage media of claim 29, further comprising instructions operable to:
providing the hearing device user with suggestions of environmental signals to record.

39. An apparatus, comprising:
one or more microphones configured to record a plurality of environmental sounds associated with at least one ambient sound environment experienced by a hearing device user;
one or more speakers; and
at least one processor configured to:
store the plurality of sounds in an environmental sound library,
provide environmental sound discrimination training to the hearing device user using a first one or more of the plurality of environmental sounds, wherein the first one or more of the plurality of environmental sounds comprise non-speech and non-musical ambient sounds, and
provide environmental sound identification training to the hearing device user using a second one or more of the plurality of environmental sounds, wherein the second one or more environmental sounds comprise non-speech and non-musical ambient sounds.

40. The apparatus of claim 39, wherein the first one or more of the plurality of environmental sounds and the second one or more of the plurality of environmental sounds are different sets of environmental sounds associated with different ambient environments.

41. The apparatus of claim 39, wherein the first one or more of the plurality of environmental sounds and the second one or more of the plurality of environmental sounds are overlapping sets of environmental sounds associated with a same ambient environment.

42. The apparatus of claim 39, wherein the one or more processors are configured to generate and provide the hearing device user with suggestions of environmental sounds to record.

43. The apparatus of claim 39, wherein the one or more processors are configured to use the at least one of the first one or more of the plurality of environmental sounds or the second one or more of the plurality of environmental sounds to provide environmental sound familiarization training to the hearing device user.

44. The apparatus of claim 43, wherein the one or more processors are configured to:
provide, to the hearing device user via a display screen, a visual display representing a selected ambient sound environment; and
while providing the visual display, deliver, to the hearing device user along with the visual display, one or more stimulation signals representing at least one of the first one or more environmental sounds or the second one or more environmental sounds.

45. The apparatus of claim 39, wherein the one or more processors are configured to:
provide, to the hearing device user via a display screen, a visual display representing a selected ambient sound environment;
while providing the visual display, deliver a sequence of stimulation signals representing at least a subset of the plurality of environmental sounds to the hearing device user, and
provide, via the display screen, at least one input for use by the hearing device user to identify a sequence of the subset of the plurality of environmental sounds.
